(19) United States
(12) Patent Application Publication
(10) Pub. No.: US 2023/0068996 A1
(43) Pub. Date: Mar. 2, 2023

(54) Formula with galactomannan gum and non digestible oligosaccharides

(71) Applicant: N.V. Nutricia, Zoetermeer (NL)

(72) Inventors: Akhtar Raish Oozeer, Utrecht (NL); Emilie Celine Rocher, Utrecht (NL)

(73) Assignee: N.V. Nutricia, Zoetermeer (NL)

(21) Appl. No.: 17/794,267

(22) PCT Filed: Jan. 28, 2021
(86) PCT No.: PCT/EP2021/051928

§ 371 Date: Jul. 21, 2022

(30) Foreign Application Priority Data
Jan. 28, 2020 (EP) 20154165.3
Oct. 7, 2020 (EP) 20200563.3

(51) Int. Cl. A23L 33/00; A23L 29/244; A23L 33/21; A23J 3/10; A61P 1/00

(52) U.S. Cl. CPC A23L 33/40; A23L 29/244; A23L 33/21; A23J 3/10; A61P 1/00

(57) ABSTRACT

The present invention relates to anti-regurgitation formula with locust bean gum and non-digestible oligosaccharides for improved effect on functional gastro-intestinal disorders in infants and young children.

### FIELD OF THE INVENTION

Field of the invention is thickened formulas for infants suffering from regurgitation

### BACKGROUND OF THE INVENTION

Breast milk is the preferred method for infant feeding. When breast feeding is not possible or the mother chooses not to breast feed, infant formula are commercially available that are suitable as a complete nutrition.

Functional gastrointestinal disorders (FGIDs) are very common during the first year of life. FGIDs are the most frequent reason for parents to consult pediatric consultations. Gastro-esophageal reflux (GER) is the backward flow of stomach contents up the esophagus and sometimes even into or out of the mouth. At the lower end of the esophagus, the lower esophageal sphincter (LES) opens when food is swallowed and then normally closes again to keep stomach contents in place. When the LES is not working properly, with GER the stomach content including hydrochloric acid, comes into contact with the esophagus, throat, nasal cavities, lungs and/or teeth, causing pain and damage. Overtime, repeated exposure of these areas with acid can cause increasing damage and cause more serious complications. The diagnostic term regurgitation is used when the reflux can be seen. According to the Rome IV criteria, regurgitation is the most prevalent FGID and has been estimated to affect as many as 30% of the infants worldwide. When the regurgitation of gastric contents causes complications or contributes to tissue damage or inflammation it is called gastro-esophageal reflux disease (GERD). GER and GERD, hereafter also referred to as GER(D), are problems especially during infancy. Besides the problems already mentioned above, GER(D) in infants may result in dehydration, impaired growth and/or a failure to thrive of the infant.

However, subjects suffering from regurgitation are often also suffering from other functional gastro-intestinal disorders (FGIDs), including problems of the lower intestinal tract, such as constipation, diarrhea, flatulence (also called gasiness), cramps and colics, see Bellaiche et al., 2018 (Acta Paediatrica 107:1276-1282). Regurgitation and GER(D) may be linked slower gastro-intestinal motility and hence constipation. It is furthermore also known that regurgitation and diarrhea are often observed in the same infant. This is possibly due to an underlying form of cow's milk protein allergy.

As in most of the cases, next to regurgitation, more than one of these symptoms occur simultaneously, there is a need infants suffering from regurgitation to be fed an infant formula that also diminishes symptoms due other FGIDs besides the regurgitation, while at the same time containing nutrients and substances needed to maintain an adequate nutritional status, proper growth and development.

US 2013/0189398 discloses infant formula specially designed to reduce episodes of regurgitation, colic and constipation that occur in infants fed infant formula. To address the regurgitation potato starch is the preferred thickening agent. Disadvantageously, starch replaces (partly) lactose as digestible carbohydrate source, which is not desired since lactose is the main source of digestible carbohydrates in human milk and has a lower glycemic index. Moreover, starch is degradable by alpha-amylase and therefore can be less efficient as thickener over time of feeding.

Savino et al., 2005 (Acta Paediatrica 94 (Suppl 449):120-124), disclose that consuming a formula with hydrolysed whey protein, starch, beta-palmitic acid and a prebiotic mixture resulted in a decrease in regurgitation in the group suffering from regurgitation upon entry of the study, and in an increase in stool frequency in the group suffering from constipation. The effects were attributed to the presence of beta-palmitic acid.

WO 2010/120172 discloses an anti-regurgitation formula which is partly fermented and contains galactomannan gum as a thickener. The effects of the viscosity in the bottle and under stomach conditions is improved.

### SUMMARY OF THE INVENTION

It was found in two clinical trials that administering an infant formula comprising galactomannan gum as thickener and non-digestible oligosaccharides to infants suffering from regurgitation resulted in, besides a further improved effect on regurgitation, beneficial effects on additional functional gastro-intestinal disorders, in particular on constipation, flatulence, diarrhea and cramps and/or colics, when compared to infants fed a similar formula without the non-digestible oligosaccharides. Also beneficial effects on stool frequency, gastro-intestinal transit time, stool consistency, crying time, abdominal pain, fussiness, abdominal distention, abdominal bloating and use of medication related to functional gastro-intestinal tract disorders. The effects were in particular observed when the formulas were partly fermented.

### DETAILED DESCRIPTION OF THE INVENTION

The present invention concerns a method for treating and/or reducing the risk of at least one functional gastro-intestinal disorder, preferably at least one functional gastro-intestinal disorder of the lower intestinal tract, in infants or young children suffering from regurgitation by administering a nutritional composition comprising galactomannan gum as thickener and non-digestible oligosaccharides to said infants.

The invention can also be worded as a nutritional composition comprising galactomannan gum as thickener and non-digestible oligosaccharides for use in infants or young children suffering from regurgitation for treating and/or reducing the risk of at least one functional gastro-intestinal disorder, preferably at least one functional gastro-intestinal disorder of the lower intestinal tract.

Alternatively the invention can be worded as galactomannan gum as thickener and non-digestible oligosaccharides comprised in a nutritional composition for use in infants or young children suffering from regurgitation for treating and/or reducing the risk of at least one functional gastro-intestinal disorder, preferably at least one functional gastro-intestinal disorder of the lower intestinal tract.

For some jurisdictions, the invention can be worded as the use of galactomannan gum as thickener and non-digestible oligosaccharides for the preparation of a nutritional composition for use in infants or young children suffering from regurgitation for treating and/or reducing the risk of at least one functional gastro-intestinal disorder, preferably at least one functional gastro-intestinal disorder of the lower intestinal tract.

The present invention also concerns a method for one or more of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders, in infants or young children suffering from regurgitation by administering a nutritional composition comprising galactomannan gum as thickener and non-digestible oligosaccharides to said infants.

The invention can also be worded as a nutritional composition comprising galactomannan gum as thickener and non-digestible oligosaccharides for use in infants or young children suffering from regurgitation for one or more of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders.

Alternatively the invention can be worded as galactomannan gum as thickener and non-digestible oligosaccharides comprised in a nutritional composition for use in infants or young children suffering from regurgitation for one or more of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders.

For some jurisdictions, the invention can be worded as the use of galactomannan gum as thickener and non-digestible oligosaccharides for the preparation of a nutritional composition for use in infants or young children suffering from regurgitation for one or more of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders.

The effects of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders are achieved when compared to infants or young children suffering from regurgitation that were adminstered at the same age for the same period of time a similar formula, hence comprising galactomannan gum as thickener, but without the non-digestible oligosaccharides. Preferably the effects are achieved when compared to infants or young children suffering from regurgitation that were adminstered at the same age for the same period of time a similar formula, hence a partly fermented formula comprising galactomannan gum as thickener, but without the non-digestible oligosaccharides.

### Regurgitation

Gastro-esophageal reflux (GER) is the backward flow of stomach contents up the esophagus. When the reflux can be seen it is diagnosed as regurgitation. In infants GER can range from reflux material simply entering the distal (bottom of the) esophagus to spitting up and possibly vomiting. Heartburn or acid reflux are also forms of GER. Gastro-esophageal Reflux disease (GERD) is a form of gastro-estophageal reflux where complications arise. GERD is a pathological process and the complications can be typical, e.g. failure to thrive, feeding and oral aversions, esophagitis, etc, or atypical, e.g. wheezing, pneumonia, chronic sinusitis, etc. Patients with GERD have complications arising from their GER that necessitate medical intervention. GERD is also referred to as “Pathogenic GER”.

Subjects suffering from regurgitation are often also suffering from other functional gastro-intestinal disorders (FGIDs), including problems of the lower intestinal tract, such as constipation, diarrhea, flatulence, cramps and colics. Bellaiche et al., 2018 (Acta Pediatrica 107:1276-1282) found in a prospective study that 14% of the infants enrolled in the study suffered from regurgitation only, next to regurgitation 4.7% also suffered from constipation, and 7.6% also suffered also from gas, and 17.4% also suffered from colics, further, next to regurgitation, 2.2% also suffered from both constipation and gas, and 2.9% also suffered from both constipation and colic, and, next to regurgitation, 5.4% also suffered from gas, constipation and colic.

Preferably the present method or uses are additionally for treating regurgitation.

### Gallactomannan Gum

The present nutritional composition comprises a galactomannan gum as a thickener. The presence of a thickener will result in an increased viscosity of the nutritional composition in the stomach and hence in a reduced regurgitation. The present nutritional composition preferably comprises at most 4 g galactomannan thickener per 100 g dry weight of the total nutritional composition, preferably at most 3 g, preferably from about 1 to 3 g, more preferably from 1.5 to 3 g, even more preferably from 2 g to 3 g galactomannan thickener per 100 g dry weight of the total nutritional composition. The present nutritional composition preferably comprises at most 1 g galactomannan thickener per 100 ml liquid nutritional composition, preferably at most 0.6 g, preferably from about 0.1 to 0.6 g galactomannan thickener per 100 ml liquid nutritional composition, more preferably from 0.2 to 0.5 g per 100 ml. A too high amount of thickener will lead to a too high viscosity, which will make bottle feeding with a teat more difficult, and a too low amount will not have a good anti-regurgitation effect.

Galactomannan gums, herein also referred to as galactomannan thickener, usually are defined as polysaccharides consisting of a mannose backbone with galactose side groups. More specifically, the present galactomannan gum can be described as a (1-4)-linked beta-D-mannan backbone with branchpoints from 6-positions linked to alpha-D-galactose, i.e. 1-6-linked alpha-D-galactose side groups. Examples of galactomannan gums are fenugreek gum, guar gum, tara gum, locust bean gum and cassia gum.

In a preferred embodiment the galactomannan thickener is locust bean gum (LBG). Locust bean gum, also referred to as carob bean gum or carobin is a product of the tree Ceratonia siliqua, or carob tree, of the family Leguminosae. The carob tree produces long pods that upon removal of the outer husk and central germ expose the layer of endosperm. It is this endosperm that is normally the source of the desired gum. A number of grades of LBG are available, and for each grade it is possible to have different particle sizes according to the requirements of the end user. In the context of the present invention, preferably LBG powder is used, wherein less than 20 wt % of the LBG particles has a particle size larger than 200 micrometer, preferably less than 10 wt % has a particle size larger than 200 micrometer.

### Non-Digestible Oligosaccharides

The present nutritional composition comprises non-digestible oligosaccharides (NDO). It was found that the presence of NDO showed a further improved anti-regurgitation effect and also improved additional functional gastro-intestinal disorders (FGIDs), in particular of the lower intestinal tract. Advantageously and most preferred, the non-digestible oligosaccharides are water-soluble (according to the method disclosed in L. Prosky et al, J. Assoc. Anal. Chem 71: 1017-1023, 1988) and preferably are oligosaccharides with a degree of polymerisation (DP) of 2 to 200. The average DP of the non-digestible oligosaccharides is preferably below 200, more preferably below 100, even more preferably below 60, most preferably below 40.

The non-digestible oligosaccharides preferably are prebiotics. Preferably the NDO are prebiotics that stimulate the growth of bifidobacteria and/or lactobacilli in the intestine. The NDO are not digested in the intestine by the action of digestive enzymes present in the human upper digestive tract. The non-digestible oligosaccharide are fermented by the human intestinal microbiota. For example, glucose, fructose, galactose, sucrose, lactose, maltose and standard maltodextrins are considered digestible. The oligosaccharide raw materials may comprise monosaccharides such as glucose, fructose, fucose, galactose, rhamnose, xylose, glucuronic acid, GalNac, etc., but these are not part of the oligosaccharides. The non-digestible oligosaccharides are preferably selected from the group consisting of fructo-oligosaccharides, non-digestible dextrins, galacto-oligosaccharides, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic acid oligosaccharides, sialyloligosaccharides and fuco-oligosaccharides, and mixtures thereof, preferably are selected from fructo-oligosaccharides and galacto-oligosaccharides, preferably are selected both. Examples of sialyloligosaccharides are 3′-sialyllactose, 6′-sialyllactose, sialyllacto-N-tetraoses, disialyllacto-N-tetraoses. Examples of fuco-oligosaccharides are (un)sulphated fucoidan oligosaccharides, 2′-fucosyllactose, 3-fucosyllactose, lacto-N-fucopentaose I, II, III, LNDH, lactodifucotetraose, lacto-N difucohexaose I and II. Preferably the present nutritional composition comprises 2′-fucosyllactose. Preferably the present nutritional composition comprises fructo-oligosaccharides and galacto-oligosaccharides. Preferably the present nutritional composition comprises fructo-oligosaccharides and galacto-oligosaccharides and 2′-fucosyllactose.

One suitable type of oligosaccharide is short-chain oligosaccharide which has an average degree of polymerisation of less than 10, preferably at most 8, preferably in the range of 2-7. The short-chain oligosaccharide preferably comprises galacto-oligosaccharides and/or fructo-oligosaccharides, for example referred to as scGOS and/or scFOS.

In one embodiment, the nutritional composition comprises galacto-oligosaccharides, preferably beta-galacto-oligosaccharides, preferably trans-galacto-oligosaccharides. The galacto-oligosaccharides preferably have an average degree of polymerisation in the range of 2-8, preferably 3-7, more in particular are short-chain oligosaccharides in the context of the invention. (Trans)galacto-oligosaccharides are for example available under the trade name Vivinal®GOS (FrieslandCampina Domo Ingredients, the Netherlands), Bimuno® (Clasado), Cup-oligo® (Nissin Sugar) and Oligomate55® (Yakult). Preferably the nutritional composition contains beta-1,6-galactosyllactose and/or beta-1,3 galactosyllactose, more preferably both.

The nutritional composition preferably comprises short-chain fructo-oligosaccharides and/or short-chain galacto-oligosaccharides, preferably at least short-chain fructo-oligosaccharides. Fructooligosaccharides may be inulin hydrolysate products having an average DP within the aforementioned (sub-) ranges; such FOS products are for instance commercially available as Raftilose® P95 (Orafti) or with Cosucra.

Another suitable type of oligosaccharide is long-chain fructo-oligosaccharides (IcFOS) which has an average degree of polymerisation above 10, typically in the range of 10-100, preferably 15-50, most preferably above 20. A particular type of long-chain fructo-oligosaccharides is inulin, such as Raftilin® HP.

The present nutritional composition may contain a mixture of two or more types of non-digestible oligosaccharides, most preferably a mixture of two non-digestible oligosaccharides. In case the NDO comprises or consists of a mixture of two distinct non-digestible oligosaccharides, one oligosaccharide may be short-chain as defined above and one oligosaccharide may be long-chain as defined above. Most preferably, short-chain oligosaccharides and long-chain oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1.

In one embodiment, the nutritional composition comprises at least two non-digestible oligosaccharides selected from of fructo-oligosaccharides and/or galacto-oligosaccharides. Suitable mixtures include a mixture of long-chain fructo-oligosaccharides with short-chain fructo-oligosaccharides or a mixture of long-chain fructo-oligosaccharides with short-chain galacto-oligosaccharides, most preferably a mixture of long-chain fructo-oligosaccharides with short-chain fructo-oligosaccharides. In one embodiment, the present composition comprises at least one non-digestible oligosaccharide other than galacto-oligosaccharides. Preferably the present composition comprises at least fructo-oligosaccharides.

The present nutritional composition preferably comprises 0.05 to 10 wt % non-digestible oligosaccharides, more preferably 0.5 to 7.5 wt %, even more preferably 1 to 5 wt %, most preferably 2 to 5 wt %, based on dry weight of the nutritional composition. When in liquid form, the nutritional composition preferably comprises 0.01 to 1.25 g non-digestible oligosaccharides, more preferably 0.05 to 1 g, even more preferably 0.25 to 0.75 g, most preferably 0.25-0.5 g, based on 100 ml nutritional composition.

### Viscosity

The nutritional compositions have a viscosity which is higher than standard infant and follow on formula due to the presence of a galactomannan gum. The increase in viscosity reduces regurgitation. The viscosity of the present nutritional composition can be determined using a starch-paddle-geometry in a concentric-cylinder with a modular compact rheometer (Physica, MCR 300, Anton Paar Benelux) at a temperature of 37° C. and at a shear rate of 10 s⁻¹. Here the viscosity relates to the viscosity of the nutritional composition that is ready to drink. Preferably, the present nutritional composition has a viscosity of at least 10 mPa·s, preferably at a temperature of 37° C. and at a shear rate of 10 s⁻¹. In one embodiment, the present nutritional composition has a viscosity of above 20 mPa·s and preferably below 300 mPa·s, and more preferably above 80 mPa·s at a temperature of 37° C. and at a shear rate of 10 s⁻¹. Preferably the viscosity is measured 15 min after reconstitution the powdered formula with water.

### Whey Protein and Casein

The present nutritional composition preferably comprises whey protein and/or casein. Preferably the present nutritional composition comprises whey protein and casein. The whey protein and/or casein in the present nutritional composition includes the whey protein and/or casein that is present in the fermented milk-derived product in case this is present.

In view of the advantageous thickening properties of the present nutritional composition, the thickening effect of coagulating casein in the stomach is not a necessity anymore. As a consequence, compared to conventional anti-reflux formula with casine/whey protein of 8/2, the amount of casein can be reduced in the nutritional composition according to the present invention. Preferably the amount based on weight of casein to whey protein ranges from 30 to 70 to 70 to 30. Preferably the present nutritional composition comprises intact casein. Preferably the present nutritional composition comprises at least 30 wt % intact casein based on total protein.

Advantageously the reduction in the amount of casein can be translated into an overall reduction of the protein content of the present nutritional composition, making it more similar to the protein content found in human milk. Hence in one embodiment, the present nutritional composition comprises less than 1.6 g protein per 100 ml, preferably less than 1.5 g. Hence, the present nutritional composition preferably comprises less than 2.5 g protein per 100 kcal, preferably less than 2.3 g per 100 kcal. In one embodiment of the present nutritional composition the sum of casein and whey protein is less than 15 wt % based on dry weight of the total nutritional composition, more preferably less than 10 wt %.

### Fermented Milk-Derived Product

The present nutritional composition preferably comprises a fermented milk-derived product or is at least partly fermented. The presence of a fermented milk-derived product together with gallactomannan thickener advantageously improves the viscosity of the product, in particular under stomach conditions, thereby further improving the anti-regurgitation effect. Furthermore, the fermented milk and the cell fragments and metabolites produced during fermentation may have a further improved effect on regurgitation and other FGID. The bioactive compounds produced during fermentation can also be referred to as postbiotics. The fermented milk-derived product can be obtained by incubation of a combination of milk, e.g. skim milk, or a milk derived product, e.g. lactose, with at least one microorganism, preferably Streptococcus thermophilus. Preferably the combination is incubated for from 10 minutes to 48 h, preferably from 2 h to 24 h, more preferably from 4 h to 12 h, even more preferably for about 6 h. A sufficient long time enables fermentation and the concomitant production of bacterial cell fragments such as glycoproteins, glycolipids, peptidoglycan, lipoteichoic acid (LTA), flagellae, lipoproteins, DNA and/or capsular polysaccharides and the production of metabolites such as lactic acid and/or galacto-oligosaccharides to take place at a sufficient or higher extent, whereas the incubation time needs not be unnecessarily long for economical reasons. The temperature during incubation is preferably between 20° C. and 50° C. After incubation, the incubated product is preferably subjected to a heat treatment. By this heat treatment preferably at least 90% of living microorganisms are inactivated. The heat treatment preferably is performed at a temperature between 80° C. and 180° C. Procedures to prepare fermented milk-derived products suitable for the purpose of the present invention are known per se. EP 778885, which is incorporated herein by reference, discloses in particular in example 7 a suitable process for preparing a suitable fermented milk-derived product. FR 2723960, which is incorporated herein by reference, discloses in particular in example 6 a suitable process for preparing a suitable fermented milk-derived product. Hence in a preferred embodiment, at least part of the nutritional composition is fermented by lactic acid producing bacteria, preferably by Streptococcus thermophilus.

Briefly, a milk-derived product, preferably pasteurised, containing lactose and optionally further macronutrients such as (vegetable) fats, casein, whey protein, vitamins and/or minerals etc. is concentrated, e.g. to between 15 to 50% dry matter and then inoculated with Streptococcus thermophilus, for example with 5% of a culture containing 10⁶ to 10¹⁰ bacteria per ml. Temperature and duration of fermentation are as mentioned above. Suitably, after fermentation the fermented milk-derived product may be pasteurised or sterilized and for example spray dried or lyophilised to provide a form suitable to be formulated in the end product further containing galactomannan thickener and NDO.

The bacterial strains of S. thermophilus that are preferably used to prepare the fermented milk-derived product for the purpose of the present develop beta-galactosidase activity in the course of fermentation of the substrate. Preferably beta-galactosidase activity develops in parallel with acidity. Preferably a beta-galactosidase activity develops which is sufficient to permit subsequent enrichment of the fermented milk-derived product in galacto-oligosaccharides. Thus preferably suitable S. thermophilus strains, when cultured on a medium containing lactose, in particular a medium based on milk concentrate, achieve fermentation of the medium accompanied by high production of galacto-oligosaccharides. Selection of a suitable strain of S. thermophilus is described in example 2 of EP 778885 and in example 1 of FR 2723960. A preferred strain of S. thermophilus is then selected that with a developing beta-galactosidase activity also produce galacto-oligosaccharides.

Preferred strains of S. thermophilus to prepare the fermented milk-derived products for the purpose of the present invention have been deposited by Compagnie Gervais Danone at the Collection Nationale de Cultures de Microorganismes (CNCM) run by the Institut Pasteur, 25 rue du Docteur Roux, Paris, France on 23 Aug. 1995 under the accession number 1-1620 and on 25 Aug. 1994 under the accession number 1-1470.

Preferably, the fermented product composition additionally is fermented by other strains of lactic acid bacteria, preferably selected from the group consisting of Lactobacillus and Bifidobacteria, more preferably Bifidobacterium breve, most preferably Bifidobacterium breve strain deposited at the CNCM under number 1-2219 at May 31, 1999.

Preferably the final nutritional composition comprises 5 to 97.5 wt. % of the fermented composition based on dry weight, more preferably 10 to 90 wt. %, more preferably 20 to 80 wt. %, even more preferably 25 to 60 wt. %. A higher concentration of fermented product above 15 wt % advantageously further improves the viscosity in the stomach. A higher concentration of fermented product above 15 wt % advantageously further reduces regurgitation and other FGIDs. As a way to specify that the final nutritional composition comprises at least partly a fermented composition, and to specify the extent of fermentation, the level of the sum of lactic acid and lactate in the final nutritional composition can be taken, as this is the metabolic end product produced by the lactic acid producing bacteria upon fermentation. The present final nutritional composition preferably comprises 0.1 to 1.5 wt. % of the sum of lactic acid and lactate based on dry weight of the composition, more preferably 0.15 to 1.0 wt. %, more preferably 0.2 to 0.8 wt. %, more preferably 0.25 to 0.8 wt. %, more preferably 0.25 to 0.5 wt. %. Alternatively the nutritional composition comprises 0.02 to 0.3 g of the sum of lactic acid and lactate per 100 kcal, preferably 0.03 to 0.2 of the sum of lactic acid and lactate per 100 kcal, preferably 0.04 to 0.15 of the sum of lactic acid and lactate per 100 kcal, preferably 0.05 to 0.15 of the sum of lactic acid and lactate per 100 kcal, preferably 0.05 to 0.1 of the sum of lactic acid and lactate per 100 kcal. Alternatively, when the composition is a liquid, the sum of lactic acid and lactate is 0.0125 to 0.2 g per 100 ml, preferably 0.02 to 0.125 g per 100 ml, preferably 0.03 to 0.10 g per 100 ml, preferably 0.035 to 0.10 g per 100 ml, preferably 0.035 to 0.07 g per 100 ml. Preferably at least 50 wt. %, even more preferably at least 90 wt. %, of the sum of lactic acid and lactate is in the form of the L(+)-isomer. Thus in one embodiment the sum of L(+)-lactic acid and L(+)-lactate is more than 50 wt. %, more preferably more than 90 wt. %, based on the sum of total lactic acid and lactate. Herein L(+)-lactate and L(+)-lactic acid is also referred to as L-lactate and L-lactic acid.

One of the postbiotic components that is preferably present is 3′-galactosyllactose. 3′-Galactosyllactose has a further improved effect on decreasing gastro-intestinal disorders of the lower intestinal tract. Hence preferably the present nutritional composition comprises 3′-galactosyllactose. 3′-Galactosyllactose can be formed by the lactase (or beta-galactosidase) enzymes of the lactic acid producing bacteria, acting on lactose during fermentation, in particular by the lactic acid bacteria mentioned above, preferably by S. thermophilus, preferably by S. thermophilus I-1620. 3′-Galactosyllactose (3′-GL) is the trisaccharide Gal-(beta 1,3)-Gal-(beta 1,4)-G1c. The nutritional composition according to the present invention preferably comprises 0.07 to 3.75 wt. % 3′-GL based on dry weight of the nutritional composition. In a preferred embodiment, the nutritional composition comprises 0.07 to 0.375 wt. % 3′-GL based on dry weight of the nutritional composition. The nutritional composition according to the present invention preferably comprises 15 to 750 mg 3′-GL per 100 kcal of the nutritional composition. In a preferred embodiment, the nutritional composition comprises 15 to 75 mg 3′-GL per 100 kcal of the nutritional composition. The nutritional composition according to the present invention preferably comprises 10 to 500 mg 3′-GL per 100 ml of the nutritional composition. In a preferred embodiment, the nutritional composition comprises 10 to 50 mg 3′-GL per 100 ml of the nutritional composition.

### Other Nutrients

The present nutritional composition preferably comprises fat in conventional amounts for anti-reflux or anti-regurgitation formula. Preferably the present nutritional composition comprises mainly vegetable fat. Preferably the present nutritional composition comprises linoleic acid (LA; 18:2 n6) and alpha-linolenic acid (ALA; 18:3 n3). Preferably the ratio LA/ALA is between 5 and 15, more preferably 5 to 10. Preferably the composition comprises LC-PUFA, more preferably docosahexaenoic acid (DHA) and/or arachidonic acid (ARA), more preferably both. The amount of the sum of DHA and ARA is preferably at least 0.4 wt % based on total fatty acids, more preferably at least 0.8 wt %. The presence of ARA and/or DHA advantageously reduces regurgitation.

In one embodiment the present nutritional composition contains less than 15% palmitic acid at the sn-2 position based on total palmitic acid and/or less than 4 wt % palmitic acid at the sn-2 position based on total fatty acids.

The present nutritional composition preferably comprises digestible carbohydrates in conventional amounts for anti-reflux or anti-regurgitation formula. Preferably the digestible carbohydrates are mainly lactose, Preferably the amount of lactose is at least 80 wt % based on total digestible carbohydrates, preferably at least 90 wt %, even more preferably at least 85 wt %. Lactose is preferred because it is closer to human milk and has a low glycemic index. In a preferred embodiment starch is not present as a thickener. Preferably the amount of starch is less than 1 wt % based on total digestible carbohydrates. Starch has a higher glycemic index than lactose, and can be degraded by amylases during feeding and can therefore be less effective in maintaining viscosity. Furthermore gallactomannan gum has a better effect on FGIDs besides regurgitation. Galactomannan gum, especially locust bean gum, may have an improved effect on the composition of the intestinal microbiota, such as stimulating the growth of Bifidobacteria which is absent with the digestible starch. An improved microbiota further reduces FGIDs of the lower intestinal tract.

### Application

The present nutritional composition is for use in infants or young children suffering from regurgitation. An infant or young child is diagnosed with regurgitation when it meets both of the following two criteria: 1) visible reflux 2 or more times per day for 3 or more weeks, and 2) no retching, hematemesis, aspiration, apnea, failure to thrive, feeding or swallowing difficulties, or abnormal posturing. The present nutritional composition is used for treating regurgitation and/or gastro-intestinal reflux disease, preferably regurgitation. It is especially advantageous to use a nutritional composition that also addresses the reduction of FGIDs of the lower intestinal tract for infants or young children that suffer from regurgitation, because these infant and young children often suffer from FGID other than regurgitation.

The present nutritional composition is preferably an infant formula, a follow-on formula or a young child formula. Examples of a young child formula are toddler milk, toddler formula and growing up milk. More preferably the nutritional composition is an infant formula or a follow-on formula. The present nutritional composition can be advantageously applied as a complete nutrition for infants. An infant formula is defined as a formula for use in infants and can for example be a starter formula, intended for infants of 0 to 6 or 0 to 4 months of age, preferably 0 to 6 months. A follow-on formula is intended for infants of 4 or 6 months to 12 months of age, preferably 6 to 12 months. At this age infants start weaning on other food. A young child formula, or toddler or growing up milk or formula is intended for children of 12 to 36 months of age. Preferably the present nutritional composition is an infant or follow on formula, preferably an infant formula.

The present nutritional composition, preferably infant formula, follow-on formula or young child formula, may be in the form of a liquid, preferably a ready-to-drink liquid, or in the form of a powder. Preferably the nutritional composition, preferably infant formula, follow-on formula or young child formula, is in the form of a powder, suitable to reconstitute with water to provide a ready-to-drink nutritional composition, preferably infant formula, follow-on formula or young child formula.

In the context of the present invention where preferred embodiments are described for the present nutritional composition, these refer to the present nutritional composition for the uses according to the invention.

The present composition is preferably administered to infants or young children with an age of 0 to 36 months, more preferably an infant with an age of 0-12 months, more preferably 0-6 months. Preferably the present composition is administered to infants with an immature LES.

The present nutritional composition is used for treating and/or reducing the risk of at least one functional gastro-intestinal disorder of the lower intestinal tract. Preferably the functional gastro-intestinal disorder is selected from a) constipation, b) flatulence, can also be indicated as gasiness, c) diarrhea and d) cramps and/or colics.

Also the present nutritional composition is used for one or more of a) increasing stool frequency, b) increasing gastro-intestinal transit time, c) improving stool consistency by decreasing the number of watery stools and hard stools, d) decreasing crying time, e) decreasing abdominal pain, f) decreasing fussiness, g) decreasing abdominal distention, h) decreasing abdominal bloating, i) decreasing medication relating to functional gastro-intestinal tract disorders.

Preferably the present nutritional composition is for treating and/or reducing the risk of flatulence. Also preferably the present nutritional composition is for treating and/or reducing the risk of diarrhea. Also preferably the present nutritional composition is for improving stool consistency by decreasing the number of watery stools and hard stools, more preferably of watery stools. Preferably the present nutritional composition is for decreasing abdominal distention.

It was found that a subgroup of the infants or young children suffering from regurgitation especially benefitted of the present nutritional composition. In particular infants or young children suffering from regurgitation that suffered from more severe gastrointestinal distress experienced a statistically more improved beneficial effect on additional functional gastro-intestinal disorders of the lower intestinal tract. Thus in one embodiment, the present nutritional composition is for use in infants or young children suffering from regurgitation wherein the infant or young child further is suffering from more severe gastrointestinal distress.

In the context of the present invention, an infant or young child that suffers from regurgitation that further suffers from more severe gastrointestinal distress is an infant of young child that has a score of 35 or more 35) determined by Infant Gastrointestinal Symptom Questionnaire (IGSQ-score) as described in Riley et al, 2015 Clinical Pediatrics 54:1167-1174.

Thus in one embodiment of the present invention, the infant or young child further suffering from more severe gastrointestinal distress, is an infant or young child having an IGSQ-score of 35 or more.

In a preferred embodiment, in the infant or young child suffering from more severe gastrointestinal distress, the frequency of hard stools is reduced compared to the frequency of hard stools in infants or young children suffering from regurgitation that were adminstered at the same age for the same period of time the same formula but that we not suffering from more severe gastrointestinal distress, wherein an infant or young child not suffering from more severe gastrointestinal distress is defined as an infant or young child having an IGSQ-score of less than 35 (<35).

This finding is particularly advantageous as especially infants or young children with increased gastrointestinal distress are more likely to be prescribed medication to relieve functional gastro-intestinal disorders of the lower intestinal tract compared to infants or young children with regurgitation.

### DESCRIPTION OF THE FIGURE

FIG. 1 shows the distribution of IGSQ sum scores for subpopulations with Baseline IGSQ sum scores ≥35(a) and with Baseline IGSQ sum scores <35 (b) in the Per-Protocol population. Box plot shows the median, Q1, Q3 and min max values, the lines show the mean; ▪=Control formula, =Test formula.

### EXAMPLES

### Example 1: Formula with Locust Bean Gum with or without Non-Digestible Oligosaccharides: Effects on Gastro-Intestinal Symptoms

A clinical trial was performed with healthy, term infants with an age less than 3.5 months that had a history of regurgitation.

As a control diet an infant formula with 0.4 g locust bean gum per 100 ml was used. As a test diet an infant formula similar to the control diet, but additionally comprising 0.8 g/100 ml non-digestible oligosaccharides (scGOS/IcFOS w/w 9:1) was used.

Both formulas had casein/whey protein in a 8/2 w/w ratio, more than 80 wt % lactose based on total digestible carbohydrates, and LC-PUFA (DHA 0.02% based on total fatty acids and ARA 0.35 wt % based on total fatty acids).

Formula was given for 8 weeks, with visits on t=0, t=2, t=4 and t=8 weeks (and a call at t=1 week).

25 infants were enrolled in the test group (ITT), and 19 infants completed the study (PP), 20% drop-out. 23 infants were enrolled in the control group (ITT), and 15 completed the study (PP), 35% drop-out.

### Results

The group that consumed the formula with locust bean gum and GOS/IcFOS showed less gastrointestinal symptoms after 8 weeks compared to the control group. The largest effect in the test group compared to the control group were mainly observed for reduced abdominal distension. This effect was statistically significantly different.

At week 4 and 8 the stool consistency in the test group was more soft formed than the control group, but the percentage of both watery stools and hard stools was less in the test group than in the control group. This is surprising, as NDO like GOS/FOS are known to reduce the consistency of stools. The test product showed a trend towards less watery and less hard/dry, but more soften stools (the latter statistically significant).

These results are indicative for an improved effect of NDO in the presence of a gallactomannan thickener on constipation, flatulence, or gasiness, diarrhea and cramps and/or colics, and also on stool frequency, gastro-intestinal transit time, stool consistency, crying time, abdominal pain, fussiness, abdominal distention, abdominal bloating, use of medication relating to functional gastro-intestinal tract disorders.

### Example 2: Reduced FGIDs in Infants Consuming Partly Fermented Formula with Locust Bean Gum as Thickener and Non-Digestible Oligosaccharides Versus Partly Fermented Formula with Locust Bean Gum but without Non-Digestible Oligosaccharides

In a clinical trial the effect of two anti-regurgitation infant formulas on FGIDs was compared. Formula 1, the control diet, was an infant formula comprising 15 wt % of the fermented formula Lactofidus™ (present on the market in France as Lactofidia from Gallia) based on dry weight (delivering about 0.17 wt % L-lactic acid) and 2.7 wt % locust bean gum based on dry weight. After reconstitution with water to a ready to drink formula, the formula comprises 0.4 g LBG per 100 ml. This formula is the same formula as the formula of example 2 of WO 2010/120172. The weight ratio casein to whey protein is 6 to 4. The amount of lactose was about 60 wt % based on total digestible carbohydrates. Formula 1 is on the market in France as Gallia A. R.

Formula 2, the test diet, was an infant formula comprising 30% fermented formula Lactofidus™ (present on the market in France as Lactofidia from Gallia) based on dry weight (delivering about 0.33 wt % L-lactic acid) and 2.7 wt % LBG and 2.7 wt % scGOS/IcFOS is an 9:1 wt ratio (source GOS VivinalGOS®, source IcFOS Raftilin® HP) based on dry weight. After reconstitution with water to a ready to drink formula the formula comprises 0.4 g LBG per 100 ml and 0.4 g scGOS/IcFOS mix. The formula also contained 0.2 wt % docosahexaenoic acid based on total fatty acids and 0.2 wt % arachidonic acid based on total fatty acids. The amount of lactose was above 95 wt % based on total digestible carbohydrates. The weight ratio casein to whey protein is 6 to 4. The amount of 3′-galactosyllactose is about 15 mg per 100 ml.

182 fully formula fed healthy term infants aged between 3-13 weeks at inclusion and diagnosed with uncomplicated regurgitation based on Rome IV criteria were included after independent ethical committee approval and registration of the study. The intervention duration was 4 weeks with optional 4 weeks extension.

At first baseline visit, t=0, baseline characteristics, anthropometrics and medical history was recorded. At visit 2, t=2 weeks, and visit 3, t=4 weeks, (serious) adverse events ((S)AE), concomittant medication and anthropometrics were recorded. On a daily base, during the first 4 weeks, stool characteristics, regurgitation severity and formula intake was registered by the parents. Gastro-intestinal symptoms (IGSQ score—Infant Gastrointestinal Symptom Questionnaire) were recorded on a weekly basis. This is a 13-item interviewer-administers questionnaire that allows parents to describe the frequency and intensity of their infants's GI signs and symptoms for the previous 7 days (Riley et al, 2015 Clinical Pediatrics 54:1167-1174). For the infants participating in the optional second part visit 4 was performed at 8 weeks, where anthropometrics, concomittant medication and (S)AE were recorded.

92 infants were enrolled in the test group, and randomized and treated. 80 completed the end visit at 4 weeks. 58 infants enrolled the optional additional phase and 55 infants completed this phase. 90 infants were enrolled in the control group, were randomized and 89 were treated. 69 infants completed the end visit at 4 weeks. 54 infants enrolled the optional additional phase and 44 infants completed this phase.

All-in-all less infants of the control group completed the trial and this was mainly due to non-serious adverse events, diarrhea being the most prevalent. However, the number of drop outs was not higher than for similar clinical trials with healthy infants.

The primary outcome parameter is the IGSQ sum-score at 4 weeks after product use in the per protocol group. An increased sum score indicates increase gastro-intestinal symptoms.

### Results

Baseline characteristics of the infants in both groups were very similar. All growth characterisitics (weight-for-age, length-for-age, weight-for-length and head circumference) were similar in both groups, and adequate growth was observed in line with the WHO z-scores for growth.

In the per protocol (PP) group the overall mean and median score of IGSQ was around 35 at the start of the intervention for both groups. Scores above 30 are considered a clinically relevant gastrointestinal tract burden. An improvement, i.e. decrease in score, to a value below 30 was observed after 1 week intervention in both groups, with a further decrease in week 2, 3 and 4. The mean and median IGSQ score was lower in the test group at t=2, 3 and 4 weeks when compared to the control. Also the decrease in mean and median IGSQ score compared to the base line was higher in the test group than in the control group.

At t=4 weeks the difference on median IGSQ score between test and control was −1.412 (s.e. 0.985), so less gastrointestinal symptoms were observed in the test group. Breaking down on individual items, the effects in the test group were observed on less difficulties of passing bowel movements, less crying time, less days that the baby was fuzzy, and less days that gas seem to make the baby uncomfortable or fussy.

The number of (serious) adverse effects ((S)AE) was higher in the control group (AE 29/92, 31.3% vs 33/89, 37.1%. SAE 3/92, 3.3% vs 7/89, 7/9%). Again, the number of (S)AE as such in the control group is not more that normally can be expected in a clinical trial like this and the type of (S)AEs were in line with what can be expected in this population. The adverse events in the control group versus test group mainly related to gastrointestinal disorders, 12.0 vs 23.6% (p=0.05). Looking at specific gastrointestinal disorders a decrease in flatulence 0 vs 5.6% (p=0.027) in the test group was the most prominent, but also occurrence of abdominal discomfort, constipation, diarrhea, dyspepsia, GERD, hard faeces, colics, vomiting, and regurgitation were reportedly lower in the test group than in the control group.

Medication related to alimentary tract and metabolism was less in the test group than in the control group (15.2% vs 20.2%). This concerned mainly drugs for acid-related disorders and drugs for functional GI-disorders (as classified by ATC therapeutic subgroup 2nd level, WHO Drug 2017/Q3).

Stool characteristics were very comparable between both groups with low incidences of watery stools or hard stools. However, in the test group the stool frequency was slightly higher than in the control group. Stool consistency was a bit lower in the test group than in the control group in the first two weeks of the study. Overall, stool frequency was however in physiological ranges and lower compared to the values observed for breastfed infants. The distribution over the different stool consistency scores was comparable between the groups, with a bit more softer stools in week 2 and 3 of the intervention group. Days were there was at least one watery stool, and days with at least 3 watery stools was a higher in the control group

These results are indicative for an improved effect of NDO in the presence of a gallactomannan thickener on constipation, flatulence, or gasiness, diarrhea and cramps and/or colics, and also on stool frequency, gastro-intestinal transit time, stool consistency, crying time, abdominal pain, fussiness, abdominal distention, abdominal bloating, use of medication relating to functional gastro-intestinal tract disorders.

### Example 3

A post hoc analysis on subpopulation level was performed on the results of the clinical trial described above in example 2. A subgroup of infants was selected with a baseline IGSQ sum score ≥35 (the overall median). From Riley et al. 2015 Clin Pediatr 54:1167 it can be concluded that scoring higher than 30-35 on the IGSQ corresponds to more concerning gastro intestinal symptom burden or more severe gastrointestinal distress, evidenced by a higher frequency and severity of stooling, spitting/up/vomiting/crying/fussiness and flatulence.

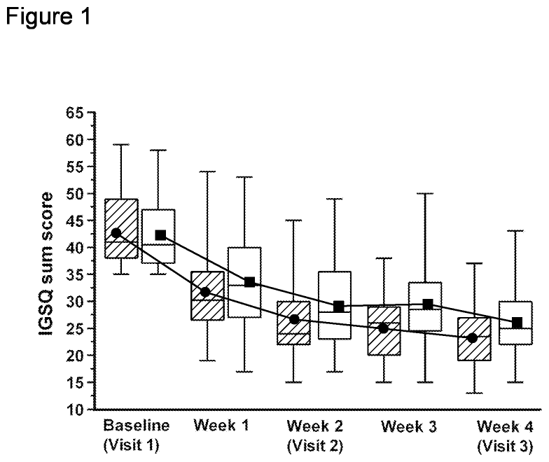

A statistically significant higher improvement of the IGSQ scores was observed in the test group versus control group (Per-Protocol population, n=82; p=0.008), using Mixed Model Repeated Measurement (MRMM) whereas this improvement was not statistically significant between study groups in the subgroup of infants with a baseline IGSQ sum score <35 (MMRM, p=0.320), see FIG. 1.

The improvement on the IGSQ score of the test group was mainly due to lower gastrointestinal tract disorders, in particular related to the IGSQ cluster stooling, a composite score, based on the individual item scores of frequency of passing hard stools during the past week (0 times in the week; 1 time in the week; 2-3 times in the week; 4-6 times in the week; 7 or more times in the week) and frequency of having difficulties with passing hard stools during the past week.

Also the cluster crying in the subpopulation of infants with baseline IGSQ sum score ≥35 seems to show a more favourable pattern over time for the test group with respect to the one of the control group.

Since especially these children with increased gastrointestinal distress are more likely to be prescribed medication, these findings are relevant for daily practice since in many cases of functional GI disorders, medications are prescribed rather than providing nutritional support.

### Statistical Analyses

The primary analysis was based on the Per-Protocol (PP) population, as defined prior to database lock. Equivalence between study groups was examined by analyzing whether the 2-sided 90% Confidence Interval (CI) of the difference in the mean IGSQ sum score at Week 4 laid within the predefined equivalence margins (α of 0.05). Data were analyzed using a linear Mixed Model Repeated Measurement (MMRM) approach on the post-baseline IGSQ sum scores with treatment, time (categorical) and interaction between treatment and time as fixed effects, baseline IGSQ sum score as adjustment covariate and center as random effect. Sensitivity analyses included the analysis of the All-Subjects-Randomised population, assessing the Missing At Random assumption and adjustment for potential covariates. An interaction test was performed to analyze the impact of the baseline IGSQ sum score on the intervention effect (predefined cut-off p-value <0.1 used to keep interaction in the model), followed by post-hoc subgroup analyses using a similar MMRM model as described above. Results were compared between groups using a Mann-Whitney Test. A Cochran-Mantel-Haenszel test was used to compare groups for the distribution over the different stool consistency categories and the incidence of diarrhea was compared based on a logistic regression model. All statistical analyses were performed using SAS Life Science Analytics Framework version 4.7.3 (SAS Institute Inc, Cary, N.C., USA).

### Example 4

Packed powdered infant formula intended for infants 6 to 12 month of age.

After reconstitution to a ready to drink formula (13.64 g powder per 100 ml) the composition comprised per 100 ml:

66 kcal

3.4 g fat (3.3 g vegetable fat; fish oil and microbial oil as source of DHA and ARA),

1.3 g protein (0.5 g whey protein; 0.8 g casein)

7.3 g digestible carbohydrates (7 g lactose)

0.375 g scGOS (of which 0.24 g has a DP 3 or higher, and 15 mg is 3′-GL) and 0.04 g IcFOS.

0.4 g locust bean gum

30% of the compostion is fermented formula (Lactofidia from Gallia) delivering about 0.45 g L-lactic acid.

The viscosity is above 10 mPa·s when measured at 10 s⁻¹ at 37° C.

On the package is indicated that the formula is an anti-regurgitation formula for treatment of regurgitation. The package further comprises at least one of the following indications: for use in prevention or treatment of mild functional gastro-intestinal disorders of the lower intestinal tract. Preferably it comprises an indication for treatment or prevention of diarrhea and/or watery stools or for use in reducing flatulence.

1.-17. (canceled)

18. A method for treating and/or reducing the risk of at least one functional gastro-intestinal disorder of the lower intestinal tract in infants or young children suffering from regurgitation by administering a nutritional composition comprising galactomannan gum as thickener and non-digestible oligosaccharides to said infants or young children.

19. A method for one or more of
a. increasing stool frequency,
b. increasing gastro-intestinal transit time,
c. improving stool consistency by decreasing the number of watery stools and hard stools,
d. decreasing crying time,
e. decreasing abdominal pain,
f. decreasing fussiness,
g. decreasing abdominal distention,
h. decreasing abdominal bloating,
i. decreasing medication relating to functional gastro-intestinal tract disorders in infants or young children suffering from regurgitation by administering a nutritional composition comprising galactomannan gum as thickener and non-digestible oligosaccharides to said infants or young children.

20.-21. (canceled)

22. The method according to claim 18, wherein the functional gastro-intestinal disorder is selected from
a. constipation,
b. flatulence,
c. diarrhea,
d. cramps and/or colics.

23. The method according to claim 18, which additionally is for treating regurgitation.

24. The method according to claim 18, wherein the non digestible oligosaccharides are selected from the group consisting of fructo-oligosaccharides, non-digestible dextrins, galacto-oligosaccharides, xylo-oligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic acid oligosaccharides, sialyloligosaccharides and fuco-oligosaccharides, and mixtures thereof.

25. The method according to claim 18, wherein the non digestible oligosaccharides are selected from the group consisting of fructo-oligosaccharides and galacto-oligosaccharides.

26. The method according to claim 18, wherein the nutritional composition comprises 0.25 to 0.75 g non-digestible oligosaccharides per 100 ml.

27. The method according to claim 18, wherein the galactomannan gum as thickener is locust bean gum.

28. The method according to claim 18, wherein the nutritional composition comprises 0.1-0.6 g galactomannan gum as thickener per 100 ml.

29. The method according to claim 18, wherein the nutritional composition comprises at least 30 wt % intact casein based on total protein.

30. The method according to claim 18, wherein at least part of the nutritional composition is fermented by lactic acid producing bacteria and wherein the nutritional composition comprises 0.25 wt % to 0.8 wt % L-lactic acid based on dry weight of the composition.

31. The method according to claim 18, wherein the nutritional composition is an infant formula, a follow on formula or a young child formula.

32. The method according to claim 18, wherein the nutritional composition comprises 3′-galactosyllactose.

33. The method according to claim 18, which is for treating and/or reducing the risk of flatulence.

34. The method according to claim 19, which is for improving stool consistency by decreasing the number of watery stools and hard stools.

35. The method according to claim 18, wherein the infant or young child is suffering from more severe gastrointestinal distress.

36. The method according to claim 35, wherein the infant or young child suffering from more severe gastrointestinal distress, is an infant or young child having an IGSQ-score of 35 or more.
